(19) United States
(12) Patent Application Publication
(10) Pub. No.: US 2023/0341548 A1
(43) Pub. Date: Oct. 26, 2023

(54) TRANSCRANIAL DOPPLER DEVICE AND SYSTEM, AND METHOD OF USE THEREOF

(71) Applicant: VIASONIX LTD, Ra’anana (IL)

(72) Inventors: Eli LEVI, Ra’anana (IL); Dan MANOR, Kadima (IL); Yarden MANOR, Tel Mond (IL); Adi YOSEF, Ra’anana (IL)

(21) Appl. No.: 18/042,379

(22) PCT Filed: Jan. 1, 2022
(86) PCT No.: PCT/IL2022/050043

§ 371 Date: Feb. 21, 2023

Related U.S. Application Data
(60) Provisional application No. 63/141,096, filed on Jan. 25, 2021.

(51) Int. Cl. G01S 7/52; G01S 15/89; A61B 8/00; A61B 8/06

(52) U.S. Cl. CPC G01S 15/8979; A61B 8/06; A61B 8/4483; G01S 7/5208; G01S 15/8911

(57) ABSTRACT

Transcranial Doppler (TCD) monitoring employing an adjustable strap system configured to fit to a head of an examined subject and having one or more TCD probe devices coupled thereto while allowing sliding motion thereover over the temporal and/or suboccipital acoustic windows of the head of the examined subject. A monitoring system is used to monitor angular positions of ultrasonic probe devices of the TCD probe devices and generate control signals to cause generation of TCD probe signals by the ultrasonic probe devices towards the acoustic windows and receive responsive TCD echo signals therethrough at the angular positions of their said ultrasonic probe devices.

### TECHNOLOGICAL FIELD

The present disclosure generally relates to Doppler ultrasonography techniques and particularly relates to a transcranial Doppler apparatus and measurement techniques.

### BACKGROUND

This section intends to provide background information concerning the present application, which is not necessarily prior art.

Ultrasound diagnosis is based on pulse-echo techniques, wherein echoes of sound waves pulses reflected inside an examined body organ, are processed and analyzed for evaluation and diagnosis. Ultrasound probes typically use piezoelectric crystals to generate very high-frequency ultrasound wave pulses by application of high-frequency electric currents. Due to the Doppler effect the frequencies of the signals reflected from moving bodies are shifted (Doppler shift) with respect to the frequencies of the original ultrasound waves generated by the probe, which enables detection of blood flow, velocity, and direction e.g., by fast Fourier transform (FFT) analysis techniques.

Transcranial Doppler (TCD) is a noninvasive and cost-effective ultrasound diagnostic technique usable for measuring in real-time cerebrovascular function, providing cerebral hemodynamics information in real-time, and detecting cerebral vessels embolization. TCD can be used to measure cerebral blood flow velocity (CBFV) and direction in the major conducting arteries of the brain (e.g., the Circle of Willis), evaluate cerebral arterial patency, detect stenosis and embolization, and profile collateral flow patterns. TCD is used nowadays in healthcare systems as a bedside tool for reliably monitoring arterial recanalization in systemic thrombolysis procedures.

In order to measure cerebral blood flow velocity (CBFV) signals the TCD probe should be placed over specific regions of the skull, known as acoustic windows, that are thin enough for the ultrasound waves to penetrate. Acoustic windows that are commonly used for TCD measurements in adults are the temporal window, orbital window, suboccipital window, and submandibular window. Because the location of these acoustic windows significantly varies from one person to another, and due to the difficulties in the identification of the CBFV waveforms that are indicative of neurological disorders, the TCD insonation (i.e., exposure to ultrasound signals) of intracranial blood vessel of interest in a subject is a difficult task.

Some TCD solutions known from the patent literature are described hereinbelow.

U.S. Pat. Publication Nos. 2016/367217 and US 2017/119347 describe a headset mountable on a head, the headset including a probe for emitting energy into the head. The headset further includes a support structure coupled to the probe. The support structure includes translation actuators for translating the probe along two axes generally parallel to a surface of the head.

U.S. Pat. Publication No. 2017/188993 describes a method for determining a neurological condition of a subject using a robotic system. The robotic system includes a transducer. The method includes determining, by a computing system, a first location with respect to a vessel of the subject, the robotic system configured to position the transducer at the first location. The method further includes receiving, by the computing system, a first signal from the vessel in response to the transducer transmitting acoustic energy towards the vessel. The method further includes analyzing, by the computing system, the received first signal to determine a first parameter of blood flow in the vessel. The method further includes determining, by the computing system, the neurological condition of the subject based on the first parameter of the blood flow in the vessel.

A headset including an interface configured to contact a head of a user in disclosed in U.S. Pat. Publication No. 2018/021021. The interface is configured to contact one or more locations along a temporal fascia region of the head of the user such that a remainder of the headset is spaced from the head of the user.

U.S. Pat. Publication No. 2018/235568 describes a transcranial Doppler-based method for displaying 3D intracranial blood flow information and a system thereof, comprising: A. performing multi-beam ultrasound scanning on predetermined intracranial areas using transcranial Doppler ultrasound probe, receiving ultrasonic echo signals; B. calculating, on basis of ultrasonic echo signal, to obtain blood flow information; C. obtaining blood flow information in a plurality of directions on basis of scanning of predetermined areas, forming stereoscopic models including blood flow information; D. visualizing data of stereoscopic model to form 3D images, and performing individualized adjustments to 3D images according to user commands, and outputting images and presenting to user.

### GENERAL DESCRIPTION

There is a need in the art for TCD measurement techniques allowing accurate acquisition, interpretation and analysis of CBFV waveforms from blood vessels within the brain. The present application discloses a robotic TCD ultrasound system configured for accurate and steady placement of robotic TCD probes over acoustic windows in the head of an examined subject, and for improved detection and mapping of blood vessels for Doppler sonography (monitoring of tissue motion and blood flow velocities, without structural imaging) and/or duplex sonography (monitoring blood flow velocities with structural imaging).

The TCD system disclosed herein comprises an adjustable headset strap system having a forehead support piece attached to an adjustable head loop, an adjustable sagittal strap attached to the forehead support piece, and one or more robotic TCD probes configured for mounting to the head loop strap while permitting sliding motion thereover (vertical and/or horizontal motion over the strap) for accurate positioning it over the temporal window or/and the suboccipital window. The head loop strap is configured to be firmly placed around the head of the examined subject by placing the forehead support piece over the forehead of the examined subject and encircling the head of the examined subject with the head loop strap, and the sagittal strap extends posteriorly from the forehead support piece and configured to loop over a posterior section of the head loop strap and firmly fit over metopic and sagittal sutures of the examined subject. This way the one or more robotic TCD probes can be manually placed firmly (i.e., immobilized) over the acoustic windows of the examined subject, and accurately positioned thereon to optimize the acquisition of the TCD signals.

The robotic TCD probes comprises in some embodiments an ultrasonic probe device mounted to a gimbal structure configured to facilitate yaw and pitch motion of the ultrasonic probe device configured to generate TCD signals. Yaw motion actuator and pitch motion actuator coupled to the gimbal structure can be used to controllably apply the yaw and/or pitch motions of the ultrasonic probe device. A respective torsion spring mechanically coupled to the rotating shaft of each one of the yaw and pitch actuators can be used for substantially cancelling backlashes (recoil) movements of the shafts when their direction of rotation is changed. In some embodiments a respective angular position sensor is coupled to the shaft of each one of the yaw and pitch actuators for generating angular position data/signals indicative of the position of the ultrasonic probe device in the generation of each TCD signal.

One inventive aspect of the subject matter disclosed herein relates to a TCD monitoring system comprising an adjustable strap system configured to fit to a head of an examined subject, one or more TCD probe devices coupled to a strap portion of the strap system while allowing sliding motion thereof over the temporal and/or suboccipital acoustic windows of the head of the examined subject, and processing means (also referred to herein as a monitoring system) configured to monitor angular positions of ultrasonic probe devices of the one or more TCD probe devices and generate control signals to cause generation of one or more TCD probe signals by said ultrasonic probe devices towards said acoustic windows, and receipt of responsive TCD echo signals therethrough at the angular positions of the ultrasonic probe devices.

The system can be configured and operable to receive and process the measurement TCD echo signals and angular position data indicative of the angular positions of the ultrasonic probe devices e.g., received from a control unit of each TCD probe device, determine for each echo signal a magnitude/strength of the echo signal at various depths, and generate based thereon a three-dimensional display indicative of blood flow in blood vessels thereby monitored i.e., indicative of/showing the cerebral blood vessels anatomy of the examined subject.

The processing means can be configured and operable to determine blood flow velocity and/or direction for each of the TCD echo signals at the various depths and generate respective blood flow velocity and/or direction data indicative thereof, and embed said blood flow velocity and/or direction data in the three-dimensional display. The processing means can be configured and operable to generate a three-dimensional display indicative of blood flow velocities in blood vessels thereby monitored. The monitoring system can be configured to issue an alarm/alert whenever measured blood flow velocity is greater than some pre-defined threshold, or smaller than another some pre-defined threshold.

In some embodiments the strap system comprises an adjustable head loop strap for encircling the head of the examined subject, a forehead support piece attached to an anterior section of the head loop strap for anchoring the head loop strap over a forehead of the examined subject, and a sagittal strap connected at one end thereof to the anterior section of the adjustable head loop strap e.g., at the forehead support piece, and adjustably looped over a posterior section of the head loop strap by a free end thereof. The strap system comprises in some embodiments a rear-head support piece attached to the posterior section of the head loop strap for anchoring the head loop strap to a rear-side of the head of the examined subject. Optionally, the strap system comprises one or more strap tightening/fastening mechanisms at a posterior portion of the head loop strap. Optionally, but in some embodiments preferably, the strap system comprises one or more strap tightening/fastening mechanisms at an anterior portion of the head loop strap.

The strap system may comprise, in addition to, or instead of, the sagittal strap, a coronal strap connected at one end thereof to a lateral section of the adjustable head loop strap and adjustably coupled to an opposite lateral section of the head loop strap by a free end thereof. Optionally, but in some embodiments preferably, the forehead support is configured to set a tilt angle of at least one of the TCD probe devices for targeting a desired area inside the head of the examined subject.

The TCD probe device comprises in some embodiments an ultrasonic probe device, a gimbal structure configured to effect pitch and yaw angular motion of the ultrasonic probe device, and pitch and yaw motion actuators for effecting the pitch and yaw angular motion. A control unit is used in some embodiments to: generate control signals for operating said yaw and/or pitch motion actuators to change angular position of the ultrasonic probe device; operating the ultrasonic probe device to generate TCD probe signals and receive TCD echo signals responsive to the TCD probe signals; and transmit the received TCD echo signals to the processing means for processing and analysis. The system comprises in some embodiments an elastic element(s) coupled to the pitch and/or yaw motion actuators to substantially prevent backlash motion of the ultrasonic probe device.

In some embodiments the system comprises angular position sensors for measuring pitch and yaw position of the ultrasonic probe device and generate angular position data indicative thereof. The TCD probe device further comprises a housing having a plurality of attachment plates configured to receive and firmly hold a portion of the adjustable head loop strap. Optionally, but in some embodiments preferably, the attachment plates are configured to permit sliding motion of the TCD probe device over the head loop strap, and after placement over the respective acoustic window, to substantially immobilize the TCD probe device at the location over the head loop strap at which the sliding motions was stopped. Optionally, but in some embodiments preferably, the attachment plates are configured to further permit a limited vertical movement allowing to vertically translate the TCD probe devices over the strap i.e., up-down adjustment movements, and thereafter substantially immobilize the TCD probe device at the adjusted vertical location over the head loop strap.

The system can comprise one or more, but in some embodiments two or more, TCD probe devices attached to the strap system, each of the two or more TCD probe devices configured for independent sliding motion over the adjustable head loop strap. The adjustable strap system can be configured accordingly to locate at least one of the TCD probe devices over a temporal window of the examined subject, and at least one other TCD probe device over a suboccipital window of the subject. In possible embodiments two of the TCD probe devices elements are placed over right and left suboccipital posterior acoustic windows of the examined subject. In some embodiments two of the TCD probe devices are placed over right and left temporal acoustic windows of the examined subject.

The processing means is configured in some embodiments to issue an alarm/alert whenever the measured blood flow velocity is greater than some pre-defined threshold, or smaller than another some pre-defined threshold. The adjustable strap system can be configured for disposal after carrying out at least one TCD measurement session therewith.

Another inventive aspect of the subject matter disclosed herein relates to a method of acquiring TCD echo signals from a head of an examined subject. The method comprising coupling one or more TCD probe devices to an adjustable strap system, adjusting a length of at least one strap of the adjustable strap system for firmly fitting it over the head of the examined subject, sliding at least one of the TCD probe devices over a strap section of the adjustable strap system for placing it over an acoustic window of the head of the examined subject, operating an ultrasonic probe device of the TCD probe device for generating TCD probe signals towards the acoustic window and for receiving responsive TCD echo signals therethrough. The adjusting may comprise adjusting the length of at least one of a sagittal or coronal strap of the adjustable strap system.

The method may comprise coupling a forehead support to the adjustable strap system for anchoring the same to the forehead of the examined subject. The method comprises in some embodiments configuring the forehead support to set a tilt angle of at least one of the TCD probe device.

The sliding comprises in possible embodiments locating at least one of the TCD probe devices over a temporal window of the examined subject and locating at least another one of the TCD probe devices over a suboccipital window of the subject. The sliding may additionally, or alternatively, comprise locating two of the TCD probe devices over right and left suboccipital posterior acoustic windows of the examined subject. In possible embodiments the sliding comprises locating two of the TCD probe devices over right and left temporal acoustic windows of the examined subject.

The method comprises in some embodiments receiving and processing the TCD echo signals and angular position data indicative of the angular positions of the ultrasonic probe devices of the TCD probe devices, determining for each of said TCD echo signals a magnitude/strength of the echo signal at various depths, and generating based thereon a three-dimensional display indicative of blood flow. The method can comprise determining blood flow velocity and/or direction for each echo signal at various depths, generating respective blood flow velocity and/or direction data indicative thereof, and embedding the blood flow velocity and/or direction data in the three-dimensional display. The method comprises in some embodiments generating an alarm/alert whenever the determined blood flow velocity is greater than some pre-defined threshold, or smaller than another some pre-defined threshold.

In some embodiments the method comprises adjusting at least one of pitch and/or yaw orientation of the ultrasonic probe device and scanning at least one area inside the head of the treated subject according to predefined scan step size. The method can further comprise measuring at least one of pitch or yaw position of the ultrasonic probe device and generating angular position data indicative thereof. Optionally, but in some embodiments preferably, the method comprises forming a feedback loop utilizing the angular position data to set a desired pitch and/or yaw orientation of the ultrasonic probe device for targeting a specific area inside the head of the examined subject. The method may accordingly comprise receiving input data indicative of the specific area inside the head of the examined subject, using the feedback loop to set the pitch and/or yaw orientation of the ultrasonic probe device for targeting the specific area, and adjusting at least one of pitch and/or yaw orientation of the ultrasonic probe device for scanning the specific area utilizing a scan step smaller than the predefined scan step size.

The method may comprise disposing the adjustable strap system after carrying out at least one TCD measurement session therewith.

Optionally, but in some embodiments preferably, at least one of the straps of the adjustable headset strap system is elastic in at least one strap section thereof, to thereby facilitate firmly fitting of the adjustable headset strap system over the head of the examined subject, and/or facilitate sideway and/or up-down sliding movement of the TCD probe devices over the adjustable head loop.

### BRIEF DESCRIPTION OF THE DRAWINGS

In order to understand the invention and to see how it may be carried out in practice, embodiments will now be described, by way of non-limiting example only, with reference to the accompanying drawings. Features shown in the drawings are meant to be illustrative of only some embodiments of the invention, unless otherwise implicitly indicated. In the drawings like reference numerals are used to indicate corresponding parts, and in which:

FIGS. 1A to 1G show a TCD headset strap system according to some possible embodiments, wherein FIG. 1A shows the TCD headset strap system in an open (spread) state, FIG. 1B shows the TCD headset strap system in the open state with a monitoring pad attached thereto, FIG. 1C shows the TCD headset strap system with the monitoring pad in an operative state, FIGS. 1D and 1E demonstrate attachment of robotic TCD probe units to the headset strap system, FIGS. 1F and 1G show the TCD headset with the robotic TCD probe units in operative states;

FIGS. 2A and 2B respectively show perspective and top views of another TCD headset strap system according to some possible embodiments;

FIGS. 3A to 3D show another configuration of the TCD headset strap system and utilization thereof in some embodiments for monitoring posterior cerebral blood flow, wherein FIG. 3A shows a top perspective view, FIG. 3B shows a back perspective view, FIG. 3C shows a top view, and FIG. 3D shows a top-back view;

FIGS. 4A to 4D demonstrate utilization of the TCD headset strap system of FIGS. 1A to 1G for multi-window monitoring application according to a possible embodiment;

FIGS. 5A and 5B schematically illustrate a TCD head strap system according to some possible embodiments utilizing a forehead support element to effect a tilt angle to at least one robotic TCD probe unit, wherein FIG. 5A shows a top view of the TCD head strap system in an operative state and FIG. 5B shows a perspective elevated side view of the TCD head strap system;

FIG. 6 schematically illustrates a robotic TCD probe unit according to some possible embodiments;

FIG. 7 is a flowchart schematically illustrating TCD monitoring process according to some possible embodiments; and

FIGS. 8A to 8I show human-machine interfaces (HMIs) of a TCD monitoring system according to some possible embodiments, wherein FIG. 8A shows a main HMI of the monitoring system, FIGS. 8B to 8D show HMIs of a quick initial signal search procedure, FIGS. 8E and 8F show HMIs of a quick low-resolution scan procedure, or a slow high-resolution, scan procedure, and FIGS. 8G to 8I show HMIs of monitoring and relocating of detected signals.

### DETAILED DESCRIPTION OF EMBODIMENTS

One or more specific and/or alternative embodiments of the present disclosure will be described below with reference to the drawings, which are to be considered in all aspects as illustrative only and not restrictive in any manner. It shall be apparent to one skilled in the art that these embodiments may be practiced without such specific details. In an effort to provide a concise description of these embodiments, not all features or details of an actual implementation are described at length in the specification. Elements illustrated in the drawings are not necessarily to scale, or in correct proportional relationships, which are not critical. Emphasis instead being placed upon clearly illustrating the principles of the invention such that persons skilled in the art will be able to make and use the TCD equipment/system, once they understand the principles of the subject matter disclosed herein. This invention may be provided in other specific forms and embodiments without departing from the essential characteristics described herein.

The present application discloses system, equipment and techniques, for carrying out real-time TCD measurements in examined subjects, monitoring cerebral blood flow, and/or mapping cerebral arteries of the examined subject. A TCD headset strap system configured for quick tight adjustment and placement over the head of the examined subject, is used in some embodiments to position one or more robotic TCD probes over respective acoustic (temporal and/or suboccipital) windows in the skull of the examined subject. The headset is configured to firmly hold and immobilize the robotic TCD probes over the acoustic windows while permitting to adjust the location of each of the robotic TCD probes by sliding movement over the straps of the headset for optimizing its placement over the respective acoustic window and improved detection of the TCD echo signals.

Each robotic TCD probe comprises a housing and an ultrasonic probe device (e.g., any suitable ultrasonic probe operating in the TCD frequencies, such as in the range of 1 to 2.5 MHz) mounted inside the housing to a gimbal structure configured to facilitate yaw and/or pitch motion of the ultrasonic doppler probe. The robotic TCD probe comprises a yaw motion actuator and a pitch motion actuator. A rotatable shaft of each actuator is coupled in some embodiments to a respective torsion spring configured to substantially prevent/minimize backlashes (e.g., out of step recoil) movement of the motor shaft. Each actuator can be coupled to a respective gear system for facilitating accurate yaw and/or pitch motion of the ultrasonic probe device under the torsion forces applied by the respective torsion spring. This way, accurate high-resolution scans of the acoustic windows of the examined subject can be achieved for detection of TCD signals, mapping the cerebral arteries, and measuring blood flow velocity and direction of the examined subject.

Optionally, but in some embodiments preferably, an angular position sensor (e.g., potentiometer) is mechanically coupled to the rotating shaft of each of the yaw and pitch motion actuators, for generating accurate yaw and pitch angular position data/signals for the echo (reflection) data/signals received at each angular position of the ultrasonic probe device. A control unit can be used in each robotic TCD probe to generate control signals for activating the yaw and/or pitch actuators of the robotic TCD probe to move the ultrasonic probe device into a new position, generate by the ultrasonic probe device TCD probe signals at its new position, receive echo (reflection) TCD signals by the ultrasonic probe device, and generate and/or record measurement data indicative of the received echo (reflection) TCD signals and of the respective yaw and/or pitch angular position data/signals generated by the yaw and/or pitch angular position sensors for the new position of the ultrasonic probe device.

A monitoring system (e.g., a smart device or computer system, such as a personal computer - PC) can be used to operate the robotic TCD probes for systematically scanning the acoustic windows of the examined subject, transmit TCD probing beams at each position of the ultrasonic probe device during the scan, process the echo TCD signals measured by the ultrasonic probe devices for each angular position of the ultrasonic probe device during the scan, generate probe data indicative of blood flow at multiple depths along the TCD probing beam, and record the generated probe data together with the respective yaw and/or pitch angular position data at each position during the scan. This way, three-dimensional images of the intracranial blood arteries can be constructed and displayed in a display of the monitoring system with suitable indications of the measured blood flow velocities and directions.

Optionally, but in some embodiments preferably, the monitoring system is configured and operable to instruct the control units of the robotic TCD probes to continuously carry out yaw and/or pitch motion of their ultrasonic probe devices over defined angular sectors, and to instruct the ultrasonic probe devices to continuously transmit TCD probe signals during their yaw and/or pitch motion, receive responsive echo TCD signals from the head of the examined subject and transmit the received echo TCD signals to the monitoring system for processing and/or recordation. The monitoring system can be configured accordingly to receive the angular position data from the control units of the robotic TCD probes and the TCD echo signals from the ultrasonic probe devices of the robotic TCD probes, associate the received TCD probe signals with respective angular position data, and process the received TCD probe signals to extract depth data at each angular position, and store the processed data obtained for each TCD echo signal with its respective angular position data.

The monitoring system can exploit the recorded yaw and pitch angular position data to interrogate specific areas within the scanned window to obtain more information about the blood flow therein. The recorded yaw and pitch angular position data can be used to conduct closed loop TCD inspection procedures to refine probe data generated at various positions. For example, but without being limiting, a specific part, or area, of a cerebral blood vessel in a displayed image can be selected by a user (e.g., using a pointing device, such as a mouse) for directing one or more ultrasonic probe devices of the robotic TCD probes to monitor in real time the specific part/area of the cerebral blood vessel. After receiving the location of the specific part/area of the cerebral blood vessel of interest in the displayed image, the monitoring system extracts from its memory the respective yaw and pitch angular position data for one or more ultrasonic probe devices of the TCD probes, and generate instructions to the control units of the respective TCD probes for closed loop monitoring of the part/area of the cerebral blood vessel of interest.

For an overview of several example features, process stages, and principles of the invention, the probe system/devices examples illustrated schematically and diagrammatically in the figures are intended for TCD ultrasonic procedures. These systems and devices are shown as one example implementation that demonstrates a number of features, processes, and principles used for TCD ultrasonic probing, but they are also useful for other applications and can be made in different variations. Therefore, this description will proceed with reference to the shown examples, but with the understanding that the invention recited in the claims below can also be implemented in myriad other ways, once the principles are understood from the descriptions, explanations, and drawings herein. All such variations, as well as any other modifications apparent to one of ordinary skill in the art and useful in diagnostic probing applications may be suitably employed, and are intended to fall within the scope of this disclosure.

An aspect of some of the embodiments disclosed herein is directed to adjustable headset strap systems configured for quick and firm accommodation over portions of head/skull of an examined subject. The disclosed adjustable headset strap systems are configured to receive and firmly hold and press one or more robotic TCD probes against acoustic windows in the head/skull of the examined subject, while permitting some degree of sideway and/or up-down sliding motion of the one or more robotic TCD probes over strap portions of the headset strap systems, for accurately adjusting them over their respective acoustic windows.

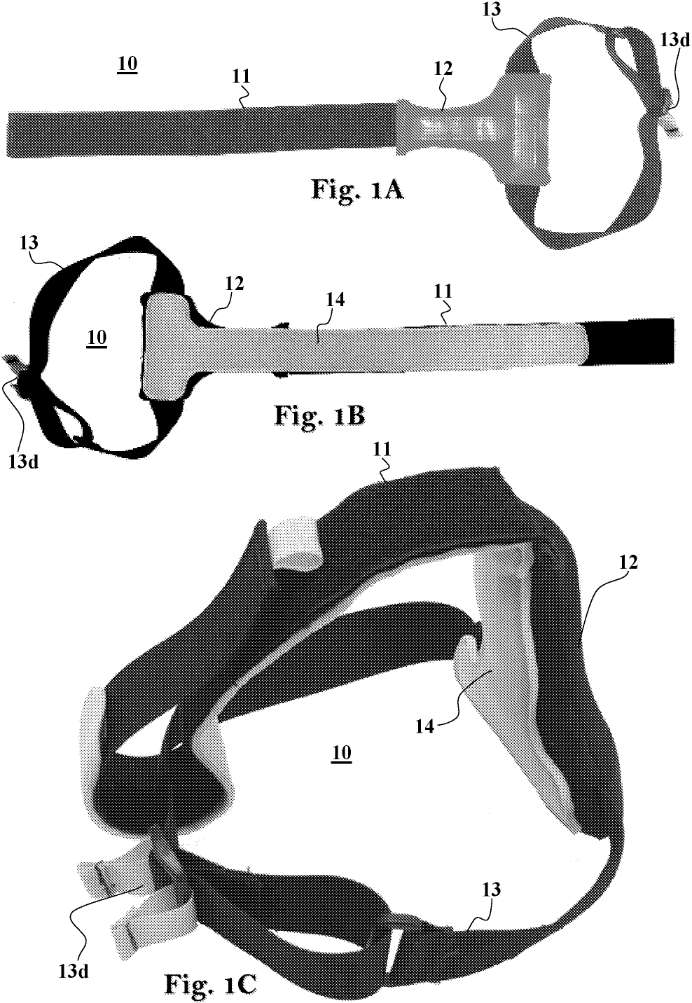

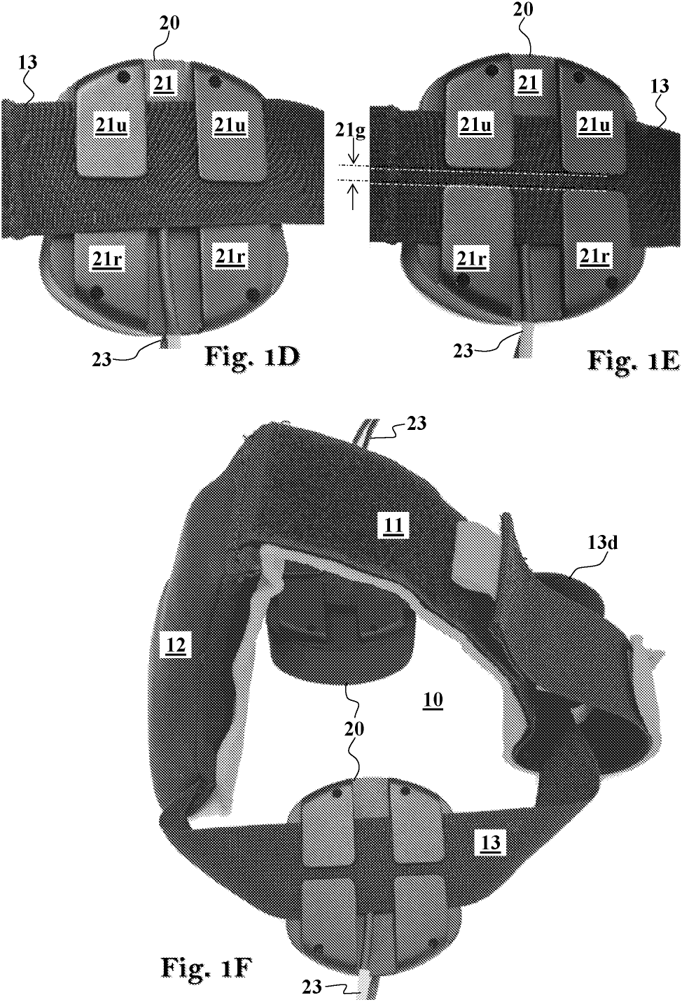

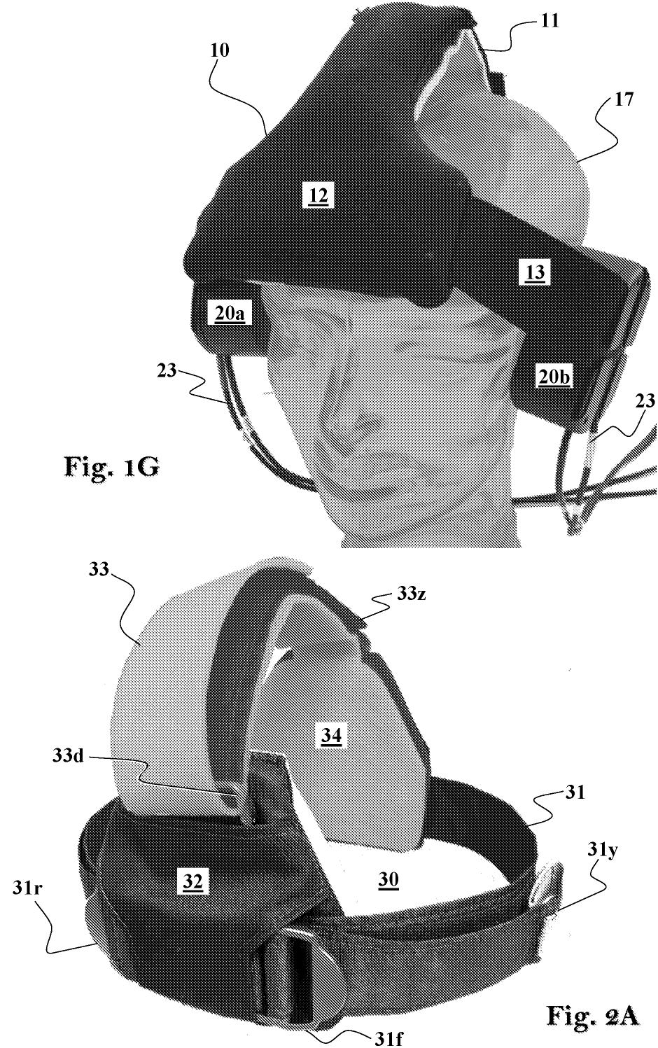

FIG. 1A shows a TCD headset 10 having a sagittal strap 11 (shown in an open/spread state) connected at one end thereof to a leg of an inverted “T″-shaped forehead support piece 12, and an adjustable head loop strap 13 extending from the arms of the inverted “T″-shaped forehead support piece 12 in opposite directions therefrom. The sagittal strap 11 in this non-limiting example comprises strap adjustment portion e.g., Velcro attachment strips, configured for looping its free end over a posterior section of the head loop strap 13 in the operational state shown in FIG. 1C. The head loop strap 13 comprises a quick strap tightening/fastening mechanism (e.g., buckle) 13d for adjusting the size of the head loop strap to firmly fit it over the head of the examined subject.

FIG. 1B shows the TCD headset 10 with a disposable monitoring pad 14 attached over internal portions of the forehead support piece 12 and a portion of the sagittal strap 11. FIG. 1C shows the TCD headset 10 in an operative state, wherein the size of the head loop strap 13 is adjusted to fit over the head of the examined subject, and the free end of the sagittal strap 11 is looped over the posterior section of the head loop strap 13 for fitting the headset 10 to the head/skull of the examined subject (not shown).

FIGS. 1D and 1E demonstrate attachment of a robotic TCD probe unit 20 to the head loop strap 13 of the headset (10). As seen, each robotic TCD probe unit 20 comprises a housing 21, and attachment plates 21u, 21r extending parallel to an external face of the housing 21 and configured to receive and firmly attach to a portion of the head loop strap 13. In this specific and non-limiting example each housing comprises two upper attachment plates 21u, and two bottom attachment plates 21r. The upper attachment plates 21u extend downwardly towards the bottom attachment plates 21r, and the bottom attachment plates 21r extend upwardly towards upper attachment plates 21u. It is noted that any other suitable number of attachment plates 21u and/or 21r can be used in the robotic TCD probe unit 20 e.g., using a single attachment plate 21u and/or 21r, and/or two, three, or more attachment plates 21u and/or 21r.

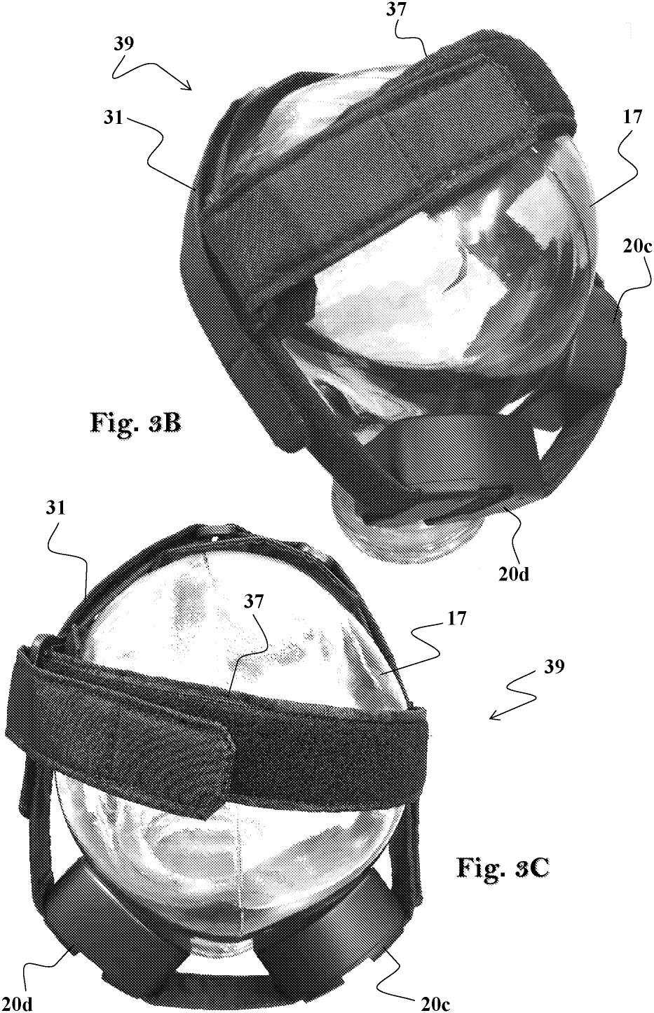

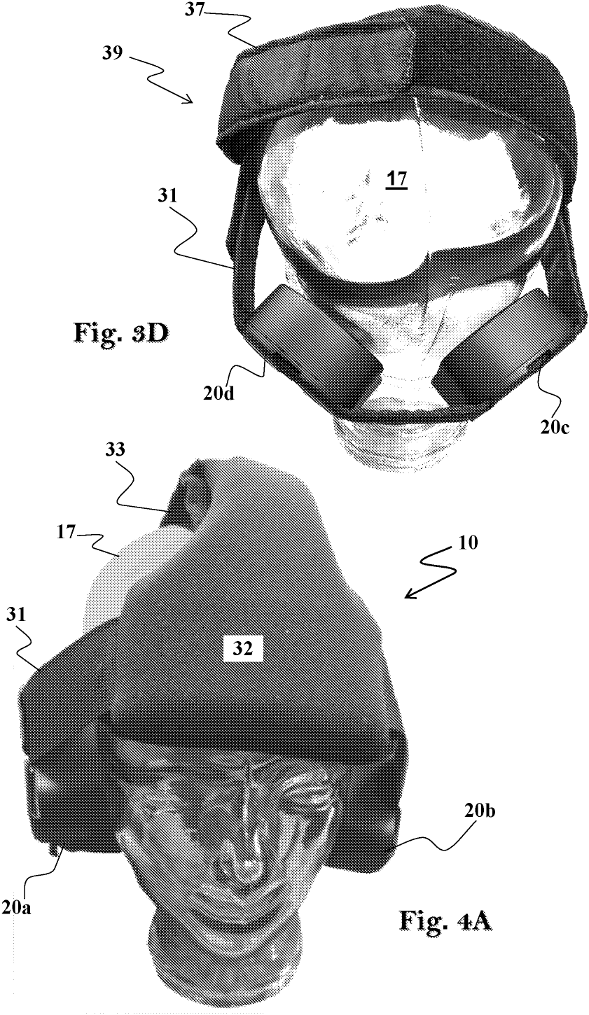

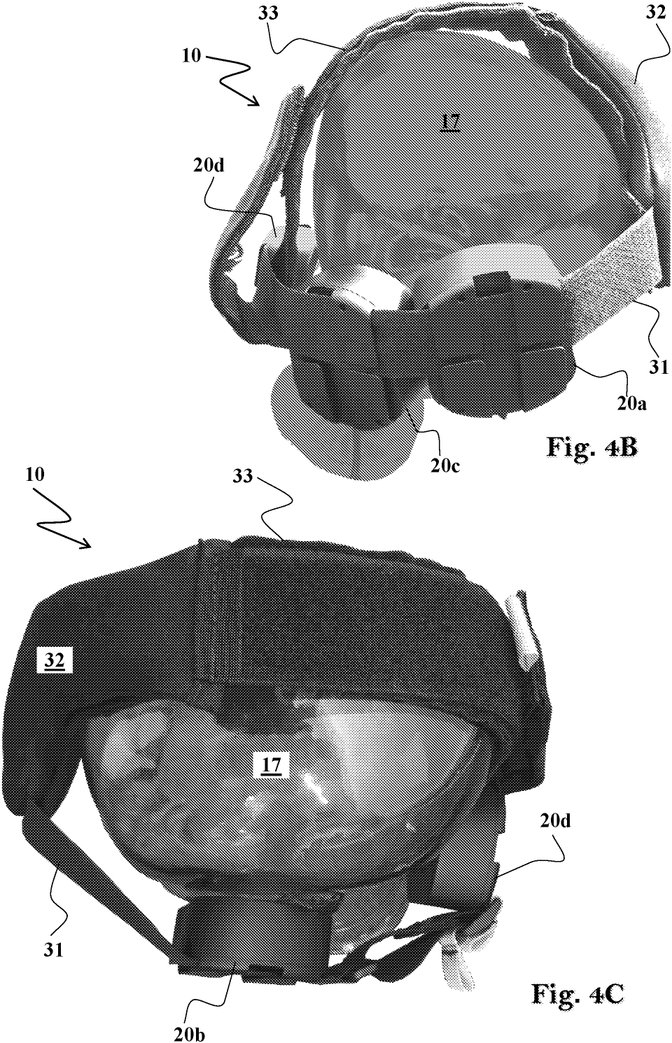

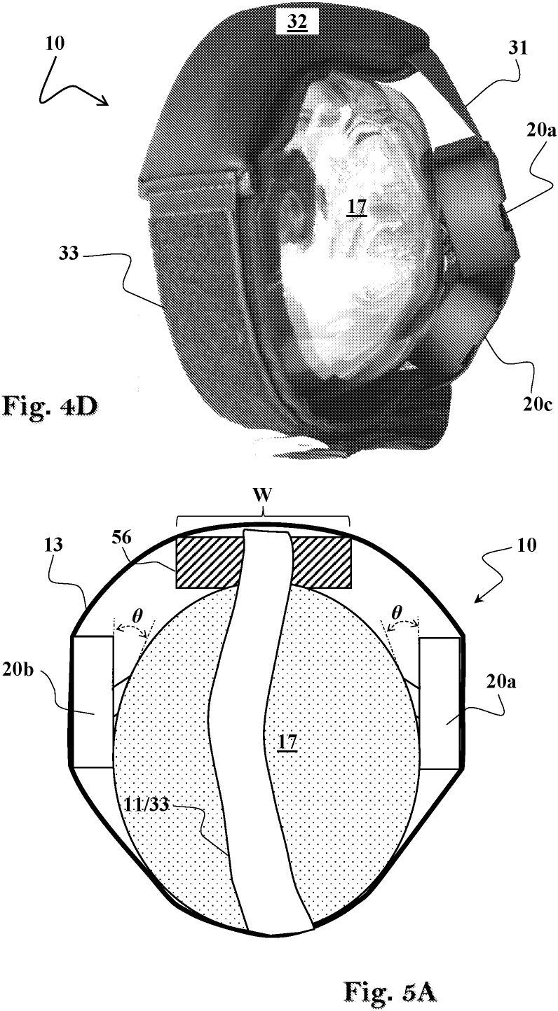

As seen, a gap 21g defined between the free ends of the upper and bottom attachment plates 21u, 21r is used for insertion of a portion of the head loop strap 13 under the attachment plates 21u, 21r. The attachment plates 21u, 21r are configured to firmly attach over the portion of the head loop strap 13 inserted therebeneath to immobilize the robotic TCD probe unit 20 thereon, while permitting some degree of sliding movement of the robotic TCD probe unit 20 for adjusting its location over the acoustic window or the examined subject after the straps of the headset 10 are adjusted over the head of the examined subject. This way, the straps of the headset 10 can press the internal face of the robotic TCD probe units 20 against the head 17 of the examined subject, while permitting sliding motion thereof over the head loop strap 13 for accurate positioning over the desired acoustic window. As better seen in FIGS. 3 and 4, the headset 10 is configured to permit sliding motion of the robotic TCD probe units 20 between temporal and suboccipital acoustic windows of the examined subject. In addition, the attachment plates 21u, 21r can be further configured to permit limited freedom to vertically translate (up-down movement) the robotic TCD probe units 20 over the head loop strap 13 for fine tuning their positions over the acoustic windows and thereby the TCD signal detection.

FIG. 1D shows the TCD probe unit 20 after a portion of the head loop strap 13 is located underneath its upper attachment plates 21u, and FIG. 1E shows the TCD probe unit 20 after the same portion of the head loop strap 13 is located underneath its bottom attachment plates 21r. As also seen in FIGS. 1D and 1E an electric cable 23 extending downwardly from the housing 21 of each robotic TCD probe unit 20, used for power supply to, and data communication with, the robotic TCD probe unit 20.

FIG. 1F shows the TCD headset 10 with the robotic TCD probe 20 units slidingly attached over the lateral portions of the head loop strap 13. In this state the headset 10 is ready for placement over the head of the examined subject. FIG. 1G shows the TCD headset 10 with two robotic TCD probe units 20a, 20b in operative state over temporal windows of a head 17. After the TCD headset 10 is placed over the head 17 with the robotic TCD probe units 20a, 20b the size of the head loop strap 13 is adjusted by the quick tightening/fastening mechanism 13d to firmly encircle the head 17 and firmly press the TCD probe units 20a, 20b against the temporal windows of the subject’ head, and the free end of the sagittal strap 11 looping over the posterior section of the head loop strap 13 is adjusted to firmly press the forehead support piece 12 against the subject’s forehead.

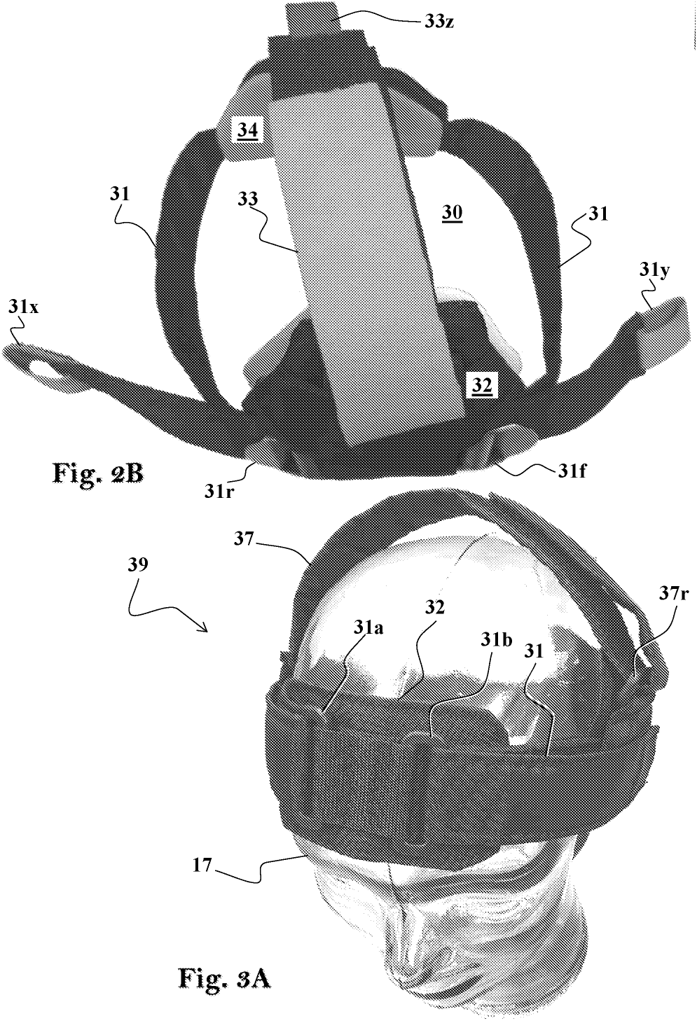

FIGS. 2A and 2B respectively show perspective and top views of another TCD headset strap system 30 according to some possible embodiments. The headset 30 comprises an adjustable head loop strap 31, and adjustable sagittal strap 33 having strap adjustment portion e.g., Velcro attachment strips, a forehead support piece 32, and a rear-head support piece 34. The forehead and read-head support pieces 32, 34 can have a tapering configuration defining a lower major/base portion and an upper minor/apex portion.

The head loop strap 31 is attached to the forehead support piece 32 by two quick tightening/fastening mechanism units (e.g., buckles) 31r, 31f attached at lateral sides of the major/base portion of the forehead support piece 32. The sagittal strap 33 is fixedly attached at one end thereof to minor/apex portion of the rear-head support piece 34, and its free end is passed through a strap retainer ring 33d attached to the minor/apex portion of the forehead support piece 32. This way, the size of the head loop strap 31 can be conveniently adjusted to the head of the examined subject by pulling and stretching its free ends 31x, 31y in opposite sideway directions through the tightening/fastening mechanism units 31r, 31f, and the size of the sagittal strap 33 can be tightly adjusted over the metopic and sagittal sutures of the examined subject’s head/skull by pulling and stretching its free end 33z through the strap retainer ring 33d and attaching it to an intermediate potion thereof by the Velcro attachment strips.

In some embodiments at least one of the straps of the adjustable headset strap system 10 and/or 30 is elastic in at least one strap section thereof, to thereby facilitate firmly fitting of the adjustable headset strap system over the head 17 of the examined subject, and/or facilitate sideway and/or up-down sliding movement of the TCD probe devices 20 over the adjustable head loop 13. For example, the head loop and/or sagittal straps of the headsets 10 and/or 30 can be prepared from a stretchable material, such as, for example, fabric (e.g., made from cotton, linen, leather, wool, polyester, nylon, olefin, vinyl, or suchlike, or any combination thereof). The forehead and/or rear-head support pieces can each have a cushion component configured to provide a soft anchor over the respective head portions against which they are pressed. The headsets 10 and/or 30 can be thus prepared from relatively inexpensive materials to produce dispensable at least partially elastic TCD headset strap systems e.g., configured for single/one-time use, and/or for a single subject, and/or for a single TCD examination/measurement session.

FIGS. 3A to 3D shows another possible TCD strap system 39 having, instead of the sagittal strap (33), an adjustable coronal strap 37 coronally extending from a right side of the head/skull 17 to its left side, or vice versa. The TCD strap system 39 comprises a forehead support piece 32 having two retainer rings 31a, 31b, and an adjustable head loop strap 31 having strap adjustment portions e.g., Velcro attachment strips. The free ends of the head loop strap 31 are looped over respective retainer rings 31a, 31b for adjusting the size of the loop and attaching its free ends to intermediate portions of the head loop strap 31. The coronal strap 37 is fixedly attached at one end thereof to one lateral portion of the head loop strap 31, and its free end is looped over a retainer ring 37r fixedly attached at another/opposite lateral portion of the head loop strap 31.

FIGS. 3B to 3D, show the TCD strap system 39 with two TCD probes 20c, 20d slidably held over a posterior portion of the head loop strap 31 for placement over suboccipital windows of the head 17. It is noted that the TCD strap systems 10 and 30 respectively shown in FIGS. 1A to 1G and FIGS. 2A to 2B, can be similarly used for placement of TCD probes over suboccipital windows of the head of an examined subject.

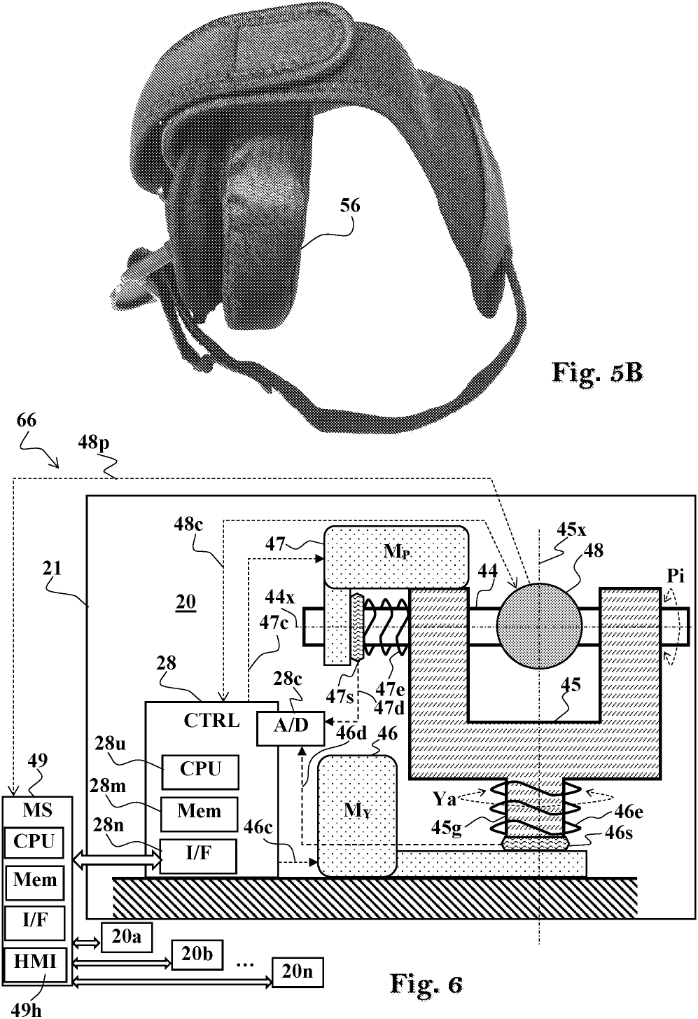

The TCD strap system 39 can be configured to include in addition to the coronal strap 37 shown in FIGS. 3A to 3D the sagittal strap 11 or 33 shown in FIGS. 1-2, and 4-5. The head loop and/or coronal straps of the headset strap system 39 can be prepared from a stretchable material, such as, for example, any type of fabric, such as mentioned hereinabove. The forehead support piece 32 can have a support element (56 in FIGS. 5A and 5B) configured to provide a soft anchor over the subject’s forehead and/or effect a tilt angle to at least one TCD probe 20. The headset strap system 39 can be thus prepared from relatively inexpensive materials to produce dispensable and at least partially elastic TCD headset strap systems e.g., configured for single/one-time use, and/or for a single subject, and/or for a single TCD examination/measurement session. At least one of the straps of the adjustable headset strap system 39 can be elastic in at least one strap section thereof.

FIGS. 4A to 4D demonstrate use of the TCD strap system 10 for concurrent multi-window monitoring applications. In these specific and non-limiting examples the ability to slidably translate the TCD probes 20 over the head loop strap 31 is exploited to place the TCD probes 20a and 20b over the temporal windows of the head/skull 17, and the TCD probes 20c and 20d over the suboccipital windows of the head/skull 17 i.e., a total of four (4) TCD probes can be used this way to simultaneously monitor cerebral blood flow of the examined subject (the TCD probes 20a, 20b, 20c and 20d, are collectively referred to herein as TCD probes 20).

The different strap systems shown in FIGS. 1 to 5 are configured in some embodiments to provide the following states: (i) an untightened state, in which the robotic TCD probe units 20 can freely slide over the head loop strap 31; and (ii) a tightened state, obtained after the head loop strap 31 is tightened, and in which the robotic TCD probe units 20 are firmly pressed by the head loop strap 31 to the head/skull of the examined subject and become substantially immobilized. It is noted that in the later state (ii), there is still some freedom to slightly slide in sideways, and/or vertically (up-down) translate, the robotic TCD probe units 20 over the head loop strap 31, thereby permitting to fine tune the position of the robotic TCD probe units 20 over their respective acoustic windows by the practitioner.

FIGS. 5A and 5B demonstrate an embodiment of the TCD strap system 10 wherein a forehead support element (e.g., a sponge, rubber and/or silicone cushion) 56 is utilized to cause a desired tilt angle θ of the TCD probe units 20a, 20b with respect to the head/skull 17 of the examined subject. The forehead support element/cushion 56 can be fixedly attached to the forehead portion of the head loop strap 13 of the TCD strap system 10, or configured to be removably connected thereto e.g., by Velcro strips. The tilt angle θ between the TCD probe units 20a, 20b and the head/skull 17 is achieved due to the width W of the forehead support element/cushion 56, which cause lateral portions of the head loop strap 13 between the forehead support element/cushion 56 and the TCD probe units 20 to extend away from head/skull 17, thereby causing a corresponding tilt of anterior portions of the TCD probe units 20 away from head/skull 17.

A main purpose of the tilt angle θ between the TCD probe units 20 and the head/skull 17 is to improve the TCD signals thereby acquired, specifically of TCD signals received from the Middle cerebral artery (MCA). Particularly, in order to effectively target the MCA some tilt angle between the TCD probe units 20 and the head/skull 17 is required from the temporal window towards the eyes of the examined subject. Different forehead support elements/cushions 56 having different widths W can be used to effect different tilt angles θ. For example, and without being limiting, in adult subjects, a forehead support element/cushion 56 having a width W of about 2 cm to 4 cm can be used to increase the tilt angle θ. The size of the forehead support element/cushion 56 can be accordingly adjusted to effect a desired tilt angle θ with examined subjects having different head circumferences/sizes.

FIG. 6 schematically illustrates a robotic TCD probe unit 20 according to some possible embodiments. The probe unit 20 comprises the housing 21, a rotatable “Y″-shaped gimbal structure 45 having an axle 44 rotatably attached to the arms of the gimbal structure 45, and an ultrasonic probe device 48 fixedly attached to the axle 44. A pitch motion actuator (and optional gear system, M_{P}) 47 mounted inside the housing 21 is used to controllably rotate the axle 44 about its longitudinal axis 44x. A yow movement actuator (and optional gear system, M_{Y}) 46 mounted inside the housing 21 is used to controllably rotate the gimbal structure 45 about the axis 45x of its leg 45g. The yaw and pitch motion actuators 46, 47 can be implemented utilizing a step, or servo electric motor. The actuators 46, 47 and their respective optional gear systems can be configured to affect rotary step, or continuous motion of ultrasonic probe device 48 about the yaw and/or pitch axes for placing the probe device 48 at a specific location/angle over the acoustic window, or for scanning a defined three-dimensional angular sector within the acoustic window.

In some embodiments an elastic element 47e is mechanically coupled to the axle 44 (and/or to a rotary shaft of the pitch motion actuator 47) for preventing and/or minimizing backlash movements of the axle 44 during changes in its direction of motion e.g., by connecting a torsion spring between the axle 44 and the gimbal structure 45. An elastic element 46e can be similarly mechanically coupled to the leg 45g of the gimbal structure 45 (and/or to a rotary shaft of the actuator) for preventing and/or minimizing backlash movements of the gimbal structure 45 during changes in its direction of motion e.g., by connecting a torsion spring between the leg 45g of the gimbal structure 45 and the housing 21.

An angular position sensor 47s (e.g., utilizing a potentiometer) is coupled in some embodiments to the axle 44 (and/or to a rotary shaft of the pitch motion actuator 47) for measuring the angular position of the axle during operation of the of the robotic TCD probe unit 20. An angular position sensor 46s (e.g., utilizing a potentiometer) can be similarly coupled to the leg 45g of the gimbal structure 45 (and/or to a rotary shaft of the yaw motion actuator 46) for measuring the angular position of the gimbal structure 45 during operation of the of the robotic TCD probe unit 20.

A control unit 28, comprising one or more processing units 28u and memories 28m is provided in some embodiments in the housing. The control unit 28 can be configured to generate control signals 48c to operate the ultrasonic probe device 48 to transmit TCD probe signals and receive responsive TCD echo signals, and control signals 47c, 46c for operating the pitch and/or yaw motion actuators 47, 46 to affect pitch and/or yaw angular movement of the ultrasonic probe device while the TCD probe signals are thereby transmitted and their respective TCD echo signals are thereby received. Optionally, but in some embodiments preferably, the ultrasonic probe device 48 is configured to transmit the TCD echo signals 48p thereby received to the monitoring system 49.

The control unit 28 can be used to receive and process the pitch and/or yaw angular position data/signals 47d, 46d generated by the angular position sensors 47s,46s (e.g., utilizing analog-to-digital converters 28c), and generate angular position data indicative thereof. The control unit can have an interface module 28n for communicating data/signals (wirelessly e.g., using WiFi, Zigbee, bluetooth, or suchlike and/or over data communication lines/bus e.g., using USB, UART, RS485, or suchlike) with one or more external devices (e.g., monitoring system - MS, computer machine, smart device) 49.

For example, in some embodiments a monitoring system (MS) 49 having one or more processors (CPU), memories (Mem), and communication modules (I/F), is used to generate control signals/data for operating a plurality the robotic TCD probe unit 20, 20a, 20b,..., 20n, to simultaneously scan different acoustic windows (e.g., one or two temporal windows and/or one or more suboccipital windows) of the examined subject, generate at each position of the ultrasonic probe devices 48 during the scans carried out by the robotic TCD probe units 20, 20a, 20b,..., 20n, respective TCD probe signals, and receive the responsive measurement data from the robotic TCD probe units 20, 20a, 20b,..., 20n i.e., receive the TCD echo data/signals 48p from the ultrasonic probe devices 48 and the respective angular (yaw and pitch) position data/signals 47d, 46d from the control units 28 of the robotic TCD probe units 20, 20a, 20b,..., 20n.

The monitoring system 49 can be configured to associate the TCD echo signals 48p received from the ultrasonic probe device 48 of each robotic TCD probe unit 20, 20a, 20b,..., 20n with the respective angular position data signals 47d, 46d received from the respective control unit 28, process the received TCD echo signals/data 48p to determine the strength/magnitude of the echo signals/data, received at each angular position of the ultrasonic probe device 48, at various different depths, and generate for each TCD probe signal respective depth data.

This way, by scanning defined yaw and pitch sectors with the ultrasonic probe devices 48 of the robotic TCD probe units 20, 20a, 20b,..., 20n, and extracting depth data for each of the received TCD echo signals at its respective angular position, a three-dimensional image of the vascular structure of the examined subject within the scanned sector is constructed, wherein each voxel of the three-dimensional image is associated with a certain depth and angular pitch and yaw position, and having a strength/magnitude of the echo TCD signal at the certain depth and angular pitch and yaw angular position. The monitoring system 49 can further associate each voxel of the three-dimensional image of the vascular structure with a respective determined blood flow velocity and/or direction, as measured by the ultrasonic probe device(s) 48 of the robotic TCD probe units 20, 20a, 20b,..., 20n.

The monitoring system 49 is configured in some embodiments to conduct closed-loop high resolution scans in smaller sections of interest within the three-dimensional image of the vascular structure. For example, the monitoring system 49 can generate control signals to operate the robotic TCD probe unit 20 to change the position of its ultrasonic probe device 48 into a certain angular position associated with a certain voxel of interest, wherein the strength/magnitude of the received TCD echo signals is relatively high (or ambiguous), generate new TCD probe signals in a small pitch/yaw sector about this certain angular position e.g., with smaller yar/pitch angular scan steps, and process the received TCD echo signals 48p to extract strengths/magnitudes of the received TCD echo signals to refine the three-dimensional image data in the vicinity of certain voxel of interest.

Closed loop monitoring sessions can be conducted by the TCD monitoring system 66, as follows:
1. At each yaw-pitch angular position of the ultrasonic probe device 48 the angular position 46d, 47d is read by the angular position sensors 46s, 47s.
2. The actuators 46, 47 are activated to rotate the ultrasonic probe device 48 into a new angular position corresponding to set a specific angular position 46d, 47d, which is read by the angular position sensors 46s, 47s.
3. The angular position 46d, 47d of the ultrasonic probe device 48 is read again by the angular position sensors 46s, 47s to obtain a new angular position reading.
4. If the obtained new angular position reading is different from the required angular position, the actuators 46, 47 are activated again to preform corrections and bring the ultrasonic probe device 48 to the exact position.
5. The angular position data/signals 46d, 47d (pitch and yaw) obtained at each step can be stored in the memory.
6. Closed loop scans can be carried out by repeating steps 2-5 until the exact selected angular position of the ultrasonic probe device 48 is obtained.

The velocity or strength/magnitude of the TCD echo signal, or a combination thereof, at each voxel of the three-dimensional image can be used to construct a color map of the cerebral blood circulation in the examined subject’s head (or at least a partial map of the cerebral vessels). Such three-dimensional mapping of the blood circulation can be exploited to identify the location of the highest measured blood flow velocity in each blood vessel within the TCD scan, which can be used to detect and/or diagnose pathologies.

The presentation of the velocity, or signal strength/magnitude data, in a three-dimensional image display can be used to provide new tools for the practitioner to explore the cerebral blood vessels. For example, after presenting the three-dimensional cerebral blood vessels map, the practitioner can move a pointing device (e.g., mouse) and select (e.g., “click”) a certain point/region of interest within the three-dimensional cerebral blood vessels map for zooming the three-dimensional display into the selected point/region of interest e.g., using strength/velocity data of previously conducted high resolution TCD probing scans, and/or using angular yaw/pitch position data associated with the certain point/region of interest to instruct the robotic TCD probe unit 20 to carry out in real-time a high resolution scan, and display a respective three-dimensional map, of the selected blood vessel about the same depth location.

It is noted that in possible embodiments the blood flow velocity at a certain voxel can be the mean (average), or peak (maximum), velocity obtained for the certain voxel over some predetermined time interval, or over a cardiac cycle, but other blood flow velocity determination techniques can be used as well/instead. In addition, strength/magnitude of the TCD echo signal at a certain voxel can be determined as the mean of a sum of spectral energy measured at the certain voxel over a defined time frame, and/or by other calculation techniques of the TCD echo signal, or of its’ m-mode display.

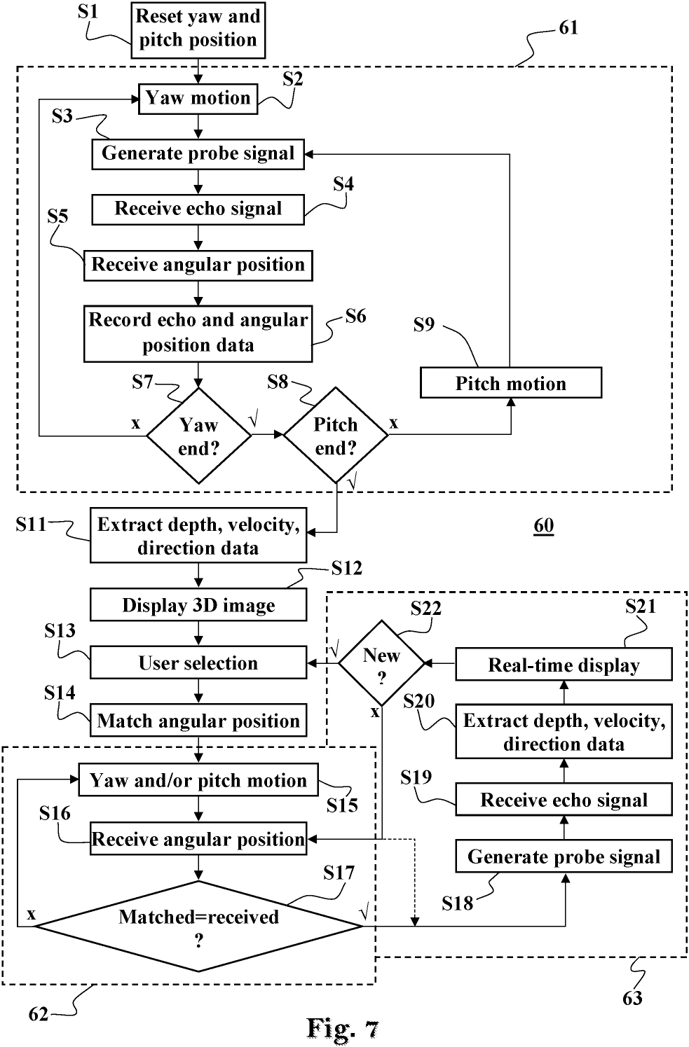

FIG. 7 is a flowchart schematically illustrating TCD monitoring processes 60 according to some possible embodiments. The process 60 can start in the resetting of the yaw and pitch position of the ultrasonic probe device (48) of the TCD probe unit (20) in step S1, for setting it into a TCD scan starting point before starting the TCD scan process 61 of steps S2 to S9.

The TCD scan process 61 can be commenced in the moving of the ultrasonic probe device (48) an incremental motion (also referred to herein as a scan step size) about the yaw axis (45x) in step S2, for generating in step S3 a TCD probe signal. The TCD probe unit (20) receives in step S4 a responsive TCD echo signal, and in step S5 the yaw and/or pitch angular position data generated by the angular position sensors (46s and 47s). The received echo signal and angular position data are processed and stored in step S6. Step S7 checks if the yaw motion of the scan is ended, and if the ultrasonic probe device (48) did not reach its yaw motion limit/range steps S2 to S7 are repeated any number of time until the yaw motion of the scan is completed.

If it is determined in step S7 that the yaw motion of the ultrasonic probe device (48) reached its scan limit/range, step S8 checks if the pitch motion of the ultrasonic probe device (48) reached its scan limit/range. If the pitch motion of the ultrasonic probe device (48) did not reach its scan limit/range, in step S9 the ultrasonic probe device (48) is moved an incremental motion (scan step size) about the pitch axis (44x) for starting a new yaw scan in steps S2 to S8 in the new pitch position, by transferring the control back to step S3. The TCD scan process 61 is ended when it is determined in step S8 that the ultrasonic probe device (48) reach its pitch motion limit.

In step S11 the stored echo data is processed to extract therefrom depth data, and respective blood flow velocity and/or direction at each depth along the recorded probe beam signal, to thereby construct a three-dimensional image of the scanned cerebral blood vessels. The three-dimensional image can be presented to a user in step S12 (e.g., in a display device), and in step S13 the user can select a specific location/area in the displayed three-dimensional image for real-time monitoring and/or zoom-in display. In step S14 the stored angular position data is scanned for finding therein angular position data matching the specific location/area selected by the user in the displayed three-dimensional image.

A closed-loop positioning process 62 can be then initiated for positioning the ultrasonic probe device (48) in the angular position matched to the user’s selected location/area in step S14. In the closed-loop positioning process 62 the ultrasonic probe device (48) is moved into a new yaw and/or pitch position in step S15, angular position data of the ultrasonic probe device (48) at its new angular position is received in step S16 from the angular position sensors (46s and 47s), for checking in step S17 if the ultrasonic probe device (48) reached matching angular position determined in step S14. If it is determined in step S17 that the angular position data received in step S16 is different from the matching angular position determined in step S14, then steps S15 to S17 are repeated any number of times required to set the position of the ultrasonic probe device (48) into the matching angular position determined in step S14.

When it is determined in step S17 that the the position of the ultrasonic probe device (48) reached the matching angular position determined in step S14, a real-time, and/or zoom-in monitoring process 63 can be carried out at the specific location/area selected by the user in step S13. The process 63 comprises generating TCD probe signal in step S18, receiving a responsive echo signal in step S19, extracting in step S20 depth data and respective blood flow velocity and/or direction data at each depth extracted along the echo signal beam, and outputting the extracted data in real-time (e.g., in a display device) in step S21. Step S22 checks if monitoring of another location/area is required by the user, and the control is passed to step S13 if a new location/area needs to be received from the user for the monitoring.

If it is determined in step S22 that further monitoring of the same location/area selection is needed, steps S16 and S17 can be carried out to ensure that the angular position of the ultrasonic probe device (48) is not changed, by receiving new angular position data from the angular position sensors (46s and 47s) and comparing it to the angular position matching the user’s selection. The closed loop position process 62 can be repeated if for any reason the angular position of the ultrasonic probe device (48) was changed. Otherwise, if the angular position of the ultrasonic probe device (48) is maintained, then steps S18 to S21 of the real-time monitoring process 63 are repeated. In possible embodiments the control can be passed from step S22 to step S18 for repeating the real-time monitoring steps if the system can guarantee that the angular position of the ultrasonic probe device (48) is maintained unchanged.

The TCD outputs provided in steps S12 and/or S21 may comprise alerts issued by the system whenever the measured blood flow velocity at a certain cerebral blood vessel is greater (or smaller) than some predefined acceptable threshold value (e.g., defined by the user via the HMI 49h).

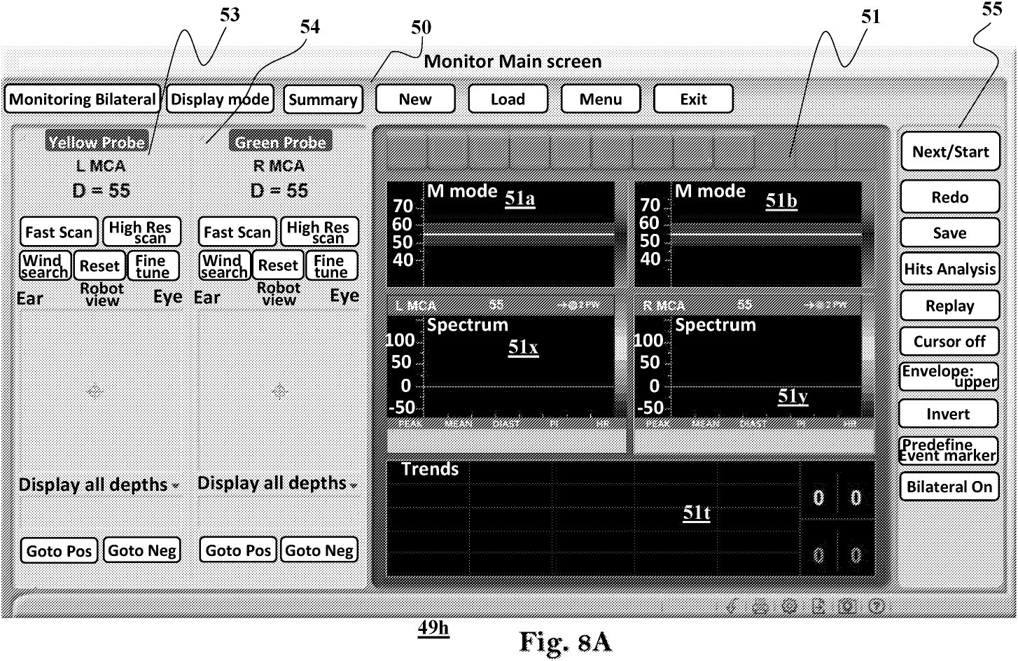

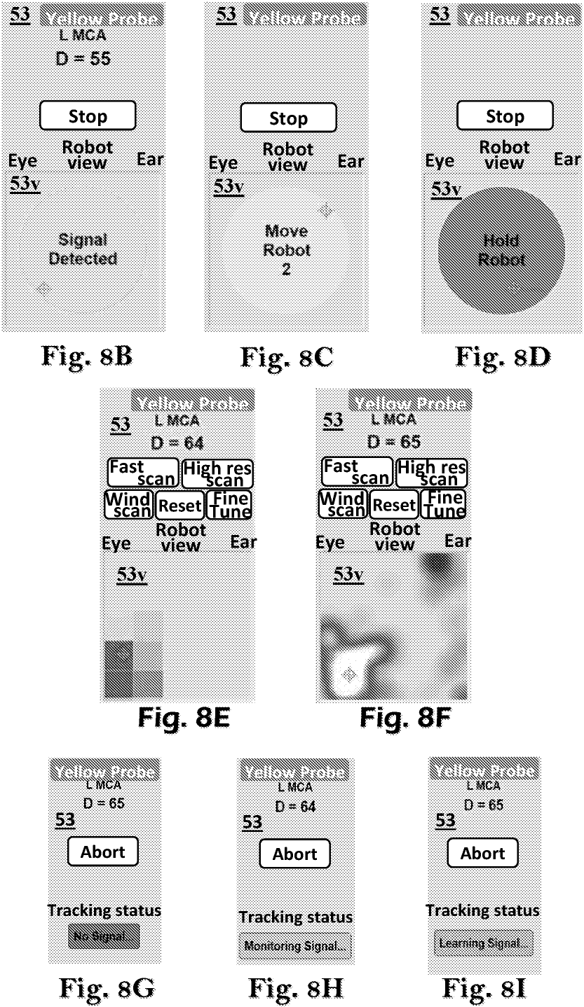

FIGS. 8A to 8I show human-machine interfaces (HMIs) of a TCD monitoring system 49 according to some possible embodiments. FIG. 8A shows a main HMI 49h of the monitoring system 49, having an operation mode bar 50, a function bar 55, a monitor display zone 51, and probe operation state selection bars 53, 54. The operation mode bar 50 comprises a bilateral monitoring press-button (Monitoring Bilateral) for toggling between bilateral and other operation modes, a Display Mode press-button selection button to toggle between the scan map display mode and the standard display mode, a Summary display press-button to show the measured waveform/trend for each monitored blood vessel, a New TCD scan press-button to enter a new patient details and start a new session protocol, a Load press-button for loading data obtained in previously conducted TCD scan sessions, a Menu press-button for software settings, and an Exit press-button for closing the HMI 49h.

The function bar 55 comprises a Next/Start press-button for starting a measurement session, Redo press-button for deleting the collected data for the specific blood vessel and reobtaining the waveform therefore, a Save press-button for saving results of the current TCD scan session, a Cursors off press-button for turning the cursors on/off, an Envelope press-button for changing the TCD curve envelope position (upper or lower), an Invert press-button for reversing the waveform direction, a Predefined events markers press-button for marking an event over the trend line, and a bilateral-on press-button for bilateral measurement.

FIG. 8A shows the display zone 51 in the bilateral display mode, having TCD M-mode measurement data displays 51a, 51b of the left and right robotic TCD probe units (20), Spectrum measurement data displays 51x, 51y of the left and right robotic TCD probe units (20), and a Trends display area 51t for displaying the velocities and parameters values over time (Mean/Peak/PI etc.). Each of the probe operation state selection bars 53, 54 comprises a window search (Wind search) press-button for preforming a quick scan to check if its positioned on the acoustic (e.g., temporal/suboccipital) window, a Fast Scan press-button for preforming the scan in large steps to find signals from the probed head region, high resolution press-button (High Res scan) for preforming the scan in smaller increments to obtain the best quality signals and perhaps to detect numerous blood vessels, a Reset press-button to reset the probe position back to a center position, and a Fine tune press-button for monitoring detected echo signals and to assist in relocating the echo signals in the event that the signal is lost.

FIGS. 8B to 8D show the probe operation state selection bar 53 of the HMI 49h during a quick initial window signal search/scan procedure. This option is designed to assist in the initial quick search for TCD echo signals in the current angular position of the ultrasonic probe device 48 in the robotic TCD probe unit 20, in order to assist in identification of the acoustic window of the examined subject. In this procedure the robotic TCD probe unit 20 is placed in a stable position in the region of the acoustic window and window search (Wind search) press-button (shown in FIG. 8A) is pressed by the practitioner. After the window search press-button is pressed, the monitoring system 49 generates control signals for the robotic TCD probe unit 20 to conduct a quick scan for TCD echo signal at the current position of the ultrasonic probe device 48. Indications concerning the quick scan results are presented in a view (Robot view) display zone 53v of the operation state selection bar 53.

In order to obtain reliable results, the robotic TCD probe unit 20 should not be moved (FIG. 8D) until the quick search/scan procedure is completed. The measurement data of the TCD echo data/signals responsive to the quick search/scan are processed by the monitoring system 49, and if no signal is detected (FIG. 8C), the monitoring system 49 instructs the practitioner to slightly move the TCD probe unit 20 (by sliding it over the head loop strap 13/31) to another position, in which the robotic TCD probe unit 20 repeats the scan automatically. If a TCD echo signal is detected (FIG. 8B) by the monitoring system 49, the Stop button is pressed by the practitioner in order to return to the main HMI screen 49h.

FIGS. 8E and 8F show the probe operation state selection bar 53 of the HMI 49h during a quick low-resolution (Fast scan) scan procedure, or a slow high-resolution (High res scan) scan procedure. These scan procedures options allow either a quick low-resolution scan, or a slower and high-resolution scan of the acoustic window of the examined subject, in order to identify a cerebral waveform, and the results of these scan procedures are displayed in the view display zone 53v. During these scan procedures the examined subject is instructed not to move or talk. The position of the ultrasonic probe device 48 is marked on the fast scan map (shown in FIG. 8E), and the high-resolution scan map (shown in FIG. 8F) e.g., with a green target icon, is displayed.

The “heat map” display shown in the view display zone 53v of the operation state selection bar 53 can be highlighted with color indications e.g., red and blue color gradients, as an indication of signal strength and blood flow direction. A pointing device (e.g., mouse) can be used to manually/automatically move the position of the ultrasonic probe device 48 to a new position e.g., by pressing/clicking on the displayed map 53v. After the scan is completed, pressing the press-buttons Go to Pos, or the Go to Neg, automatically move the position of the ultrasonic probe device 48 to the location of the strongest forward blood flow signal, or to the strongest reverse blood flow signal, respectively.

FIGS. 8G to 8I show the probe operation state selection bar 53 of the HMI 49h signal tracking procedures. This feature can be used after an echo signal is detected, in order to start learning the detected TCD echo signal, as shown FIG. 8I. The Monitoring Signal message shown in FIG. 8H appears in the selection bar 53 after the system finished to “learn” the received TCD echo signal. As shown in FIG. 8G, if the TCD echo signal is lost, a No Signal indication appears in the selection bar 53 and the system will try to relocate the TCD signal.

The learning process performed by the system comprises in some embodiments the following steps:
1. Detecting TCD echo signal indicative of blood flow properties (e.g., velocity and/or direction).
2. Determine a score to the detected TCD echo signal based at least partially on its strength.
3. If during the monitoring the strengths of newly detected TCD echo signals is deducted by more than some predefined percentage (X%), then another scan is performed around the same position to try and relocate the lost signal.

Terms such as top, bottom, upper, lower, front, back, right, and left and similar adjectives in relation to orientation of the probes/system and components of it refer to the manner in which the illustrations are positioned on the paper, not as any limitation to the orientations in which the apparatus can be used in actual applications.

It should also be understood that throughout this disclosure, where a process or method is shown or described, the steps of the method may be performed in any order or simultaneously, unless it is clear from the context that one step depends on another being performed first.

As described hereinabove and shown in the associated figures, the present invention provides systems/devices for TCD probing/monitoring and related methods. While particular embodiments of the invention have been described, it will be understood, however, that the invention is not limited thereto, since modifications may be made by those skilled in the art, particularly in light of the foregoing teachings. As will be appreciated by the skilled person, the invention can be carried out in a great variety of ways, employing more than one technique from those described above, all without exceeding the scope of the claims.

1. A transcranial Doppler (TCD) monitoring system comprising an adjustable strap system configured to fit to a head of an examined subject, one or more TCD probe devices coupled to a strap portion of said strap system while allowing sliding motion thereof over temporal and/or suboccipital acoustic windows of the head of the examined subject, and a monitoring system configured to monitor angular positions of ultrasonic probe devices of the one or more TCD probe devices and generate control signals to cause generation of TCD probe signals by said ultrasonic probe devices towards said acoustic windows, and receipt of responsive TCD echo signals therethrough at the angular positions of said ultrasonic probe devices.

2. The system of claim 1 wherein the monitoring system is configured to receive and process the TCD echo signals and angular position data indicative of the angular positions of the ultrasonic probe devices, determine for each echo signal a magnitude/strength of the echo signal at various depths, and generate based thereon a three-dimensional display indicative of blood flow in blood vessels thereby monitored.

3. The system of claim 2 wherein the monitoring system is configured to determine blood flow velocity and/or direction for each of the TCD echo signals at various depths and generate respective blood flow velocity and/or direction data indicative thereof, and embed said blood flow velocity and/or direction data in the three-dimensional display.

4. The system of claim 3 wherein the monitoring system is configured to generate a three-dimensional display indicative of blood flow velocities in blood vessels thereby monitored.

5. The system of claim 3 wherein the monitoring system is configured to issue an alarm/alert whenever measured blood flow velocity is greater than some pre-defined threshold, or smaller than another some pre-defined threshold.

6. The system of claim 1 wherein the strap system comprises an adjustable head loop strap for encircling the head of the examined subject, a forehead support attached to an anterior section of said head loop strap for anchoring said head loop strap over a forehead of the examined subject, and a sagittal strap connected at one end thereof to the anterior section of the adjustable head loop strap at the forehead support and adjustably looped over a posterior section of the head loop strap by a free end thereof.

7. The system of claim 6 wherein the strap system comprises a rear-head support piece attached to the posterior section of the head loop strap for anchoring the head loop strap to a rear-side of the head of the examined subject.

8. The system of claim 6 wherein the strap system comprises one or more strap tightening/fastening mechanisms at a posterior portion of the head loop strap.

9. The system of claim 6 wherein the strap system comprises one or more strap tightening/fastening mechanisms at an anterior portion of the head loop strap.

10. The system of claim 6 wherein the strap system comprises in addition to, or instead of, the sagittal strap, a coronal strap connected at one end thereof to a lateral section of the adjustable head loop strap and adjustably coupled to an opposite lateral section of the head loop strap by a free end thereof.

11. The system of claim 6 wherein the forehead support is configured to set a tilt angle of at least one of the TCD probe devices for targeting a desired area inside the head of the examined subject.

12. The system of claim 1 wherein the TCD probe device comprises a gimbal structure configured for pitch and yaw angular motion of said ultrasonic probe device, and pitch and yaw motion actuators configured to effect said pitch and yaw angular motion.

13. The system of claim 12 wherein the TCD probe device comprises a control unit configured to generate control signals for operating said yaw and/or pitch motion actuators to change angular position of the ultrasonic probe device, operating said ultrasonic probe device to generate TCD probe signals and receive TCD echo signals responsive to said TCD probe signals, and to transmit the received TCD echo signals to the monitoring system for processing said TCD echo signals.

14. The system of claim 13 comprising an elastic element coupled to the pitch and/or yaw motion actuators to substantially prevent backlash motion.

15. The system of claim 13 comprising angular position sensors for measuring pitch and yaw position of the ultrasonic probe device and generating angular position data indicative thereof.

16. The of claim 13 The system claim 13 comprising a housing having a plurality of attachment plates configured to receive and firmly hold a portion of the adjustable head loop strap.

17. The system of claim 1 wherein the adjustable strap system is configured to locate at least one of the TCD probe devices over a temporal window of the examined subject, and at least another one of the TCD probe devices over a suboccipital window of said subject.

18. The system of claim 1 wherein the adjustable strap system is configured to locate two of the TCD probe devices over right and left suboccipital posterior acoustic windows of the examined subject.

19. The system of claim 1 wherein the adjustable strap system is configured to locate two of the TCD probe devices over right and left temporal acoustic windows of the examined subject.

20. The system of claim 1 wherein the adjustable strap system is configured for disposal after carrying out at least one TCD measurement session therewith.

21. A method of acquiring TCD echo signals from a head of an examined subject, the method comprising: coupling one or more TCD probe devices to an adjustable strap system; adjusting a length of at least one strap of said adjustable strap system for firmly fitting it over the head of the examined subject; sliding at least one of said TCD probe devices over a strap section of said adjustable strap system for placing it over an acoustic window of the head of the examined subject, operating an ultrasonic probe device of said TCD probe device for generating TCD probe signals towards said acoustic window and for receiving responsive TCD echo signals therethrough.

22. The method of claim 21 wherein the adjusting comprises adjusting the length of at least one of a sagittal or coronal strap of the adjustable strap system.

23. The method of claim 21 comprising coupling a forehead support to the adjustable strap system for anchoring the same to the forehead of the examined subject.

24. The method of claim 23 comprising configuring the forehead support to set a tilt angle of at least one of the TCD probe device.

25. The method of claim 21 wherein the sliding comprises locating at least one of the TCD probe devices over a temporal window of the examined subject and locating at least another one of the TCD probe devices over a suboccipital window of said subject.

26. The method of claim 21 wherein the sliding comprises locating two of the TCD probe devices over right and left suboccipital posterior acoustic windows of the examined subject.

27. The method of claim 21 wherein the sliding comprises locating two of the TCD probe devices over right and left temporal acoustic windows of the examined subject.

28. The method of claim 21 comprising receiving and processing the TCD echo signals and angular position data indicative of the angular positions of the ultrasonic probe devices of the TCD probe devices, determining for each of said TCD echo signals a magnitude/strength of the echo signal at various depths, and generating based thereon a three-dimensional display indicative of blood flow.

29. The method of claim 28 comprising determining blood flow velocity and/or direction for each echo signal at various depths, generating respective blood flow velocity and/or direction data indicative thereof, and embedding said blood flow velocity and/or direction data in the three-dimensional display.

30. The method of claim 29 comprising generating an alarm/alert whenever the determined blood flow velocity is greater than some pre-defined threshold, or smaller than another some pre-defined threshold.

31. The method of claim 21 comprising adjusting at least one of pitch and/or yaw orientation of the ultrasonic probe device and scanning at least one area inside the head of the treated subject according to predefined scan step size.

32. The method of claim 31 comprising measuring at least one of pitch or yaw position of the ultrasonic probe device and generating angular position data indicative thereof.

33. The method of claim 32 comprising forming a feedback loop utilizing the angular position data to set a desired pitch and/or yaw orientation of the ultrasonic probe device for targeting a specific area inside the head of the examined subject.

34. The method of claim 33 comprising receiving input data indicative of the specific area inside the head of the examined subject, using the feedback loop to set the pitch and/or yaw orientation of the ultrasonic probe device for targeting said specific area, and adjusting at least one of pitch and/or yaw orientation of the ultrasonic probe device for scanning said specific area utilizing a scan step smaller than said predefined scan step size.

35. The method of claim 21 comprising disposing the adjustable strap system after carrying out at least one TCD measurement session therewith.
